(19) United States
(12) Patent Application Publication
(10) Pub. No.: US 2006/0252070 A1
(43) Pub. Date: Nov. 9, 2006

(54) Multi-Color Light Detection With Imaging Detectors

(75) Inventors: Steven J. Boege, San Mateo, CA (US); Christopher B. Lynch, San Jose, CA (US); Mark F. Oldham, Los Gatos, CA (US)

Correspondence Address: MILA KASAN, PATENT DEPT.;APPLIED BIOSYSTEMS, 850 LINCOLN CENTRE DRIVE, FOSTER CITY, CA, 94404, US

(73) Assignee: Applera Corporation, Foster City, CA (US)

(21) Appl. No.: 11/380,822

(22) Filed: Apr. 28, 2006

Related U.S. Application Data
(60) Provisional application No. 60/676,488, filed on Apr. 28, 2005.

(51) Int. Cl. C12Q 1/68; G06K 9/00

(52) U.S. Cl. 435/6; 382/128

(57) ABSTRACT

Methods for biological sample multi-color light detection using color-image detectors with pixel filter arrays or multi-color pixels.

### PRIORITY REFERENCE

This application claims a priority benefit under 35 U.S.C. § 119(e) from U.S. Provisional Application No. 60/676,488, filed Apr. 28, 2005, which is incorporated by reference in its entirety herein.

### FIELD

The present teachings relate to methods for multi-color light with imaging detectors.

### BACKGROUND

Fluorescence detection can include monochromatic image detectors to provide detection of fluorescence by each sample analyzed. Light emitted by samples can be separated into spectrally distinct components before reaching the image detector to determine relative emission rates from two or more sample constituents having different emission spectra. The separation can include optical diffraction, dispersion, and/or transmission filters to separate the different emission spectra. The transmission filters positioned between the emission light and the image detector can be of larger area than both the sample and image detector adding to cost. Positioning different filters between the emission light and image detector can include a system to exchange the filters with mechanical and electrical components adding to complexity. It can be desirable to replace transmission filters and associated mechanical components by detecting the emission light with a color-imaging detector.

### SUMMARY

In various embodiments, the present teachings can provide a method for detection for a biological sample including exciting multiple luminescent dyes that produce emission light in relation to nucleic acids present in the biological sample, and detecting the emission light with a multi-color detector, wherein the detector provides dedicated sections of the detector corresponding to the multiple dyes.

In various embodiments, the present teachings can provide a method for detection for a biological sample including exciting multiple luminescent dyes that produce emission light in relation to nucleic acids present in the biological sample, and detecting the emission light with a multi-color detector, wherein the method does not include filtering the emission light with a transmission filter for selecting luminescence wavelengths, and detecting the luminescence wavelengths on a monochromatic detector.

Additional embodiments are set forth in part in the description that follows, and in part will be apparent from the description, or may be learned by practice of the various embodiments described herein.

### BRIEF DESCRIPTION OF THE DRAWINGS

Various embodiments of the present teachings are exemplified in the accompanying drawings. The teachings are not limited to the embodiments depicted, and include equivalent structures and methods as set forth in the following description and known to those of ordinary skill in the art. In the drawings:

FIG. 1A-1B illustrate a view of a pixel filter array for detection of emission light from an array of biological samples including luminescent dyes that can produce emission light in relation to nucleic acid present in the biological sample according to various embodiments of the present teachings; and

FIGS. 2A-2B illustrate a view of a filter array for detection of emission light from a capillary with biological samples including luminescent dyes that can produce emission light in relation to nucleic acid present in the biological sample according to various embodiments of the present teachings;

It is to be understood that both the foregoing general description and the following detailed description are exemplary and explanatory only and are intended to provide a further explanation of the various embodiments of the present teachings.

### DESCRIPTION OF VARIOUS EMBODIMENTS

In this application, the use of the singular includes the plural unless specifically stated otherwise. In this application, the use of “or” means “and/or” unless stated otherwise. Furthermore, the use of the term “including”, as well as other forms, such as “includes” and “included”, is not limiting. Also, terms such as “element” or “component” encompass both elements and components comprising one unit and elements and components that comprise more than one subunit unless specifically stated otherwise. Wherever possible, the same reference numbers will be used throughout the drawings to refer to the same or like parts.

The section headings used herein are for organizational purposes only, and are not to be construed as limiting the subject matter described. All documents cited in this application, including, but not limited to patents, patent applications, articles, books, and treatises, are expressly incorporated by reference in their entirety for any purpose.

The term “color-imaging detection” as used herein refers to using any component, portion thereof, or system of components that can detect colored light including a charged coupled device (CCD), back-side-thinned, cooled CCD, front-side illuminated CCD, a CCD array, a photodiode, a photodiode array, a photo-multiplier tube (PMT), a PMT array, complimentary metal-oxide semiconductor (CMOS) sensors, CMOS arrays, a charge-injection device (CID), CID arrays, etc. The imaging detector can be adapted to relay information to a data collection device for storage, correlation, and/or manipulation of data, for example, a computer, or other signal processing system. In various embodiments, a color-imaging detector can be a pixel filter array imaging detector as described in U.S. Pat. No. 6,756,618. In various embodiments, the pixel filter array can include a dielectric thin film coating providing sharper cut-offs and higher transmission than would be realized with filters made from dyed photoresists. In various embodiments, a color-imaging detector can be multi-color pixel imaging detector as described in U.S. Pat. No. 5,965,875.

The term “sample chamber” as used herein refers to any structure that provides containment to a sample. The sample chamber can be open or transparent to provide entry to excitation light and egress to fluorescent light. The transparency can be provided by glass, plastic, fused silica, etc. The sample chamber can take any shape including a well, a tube, a vial, a cuvette, a tray, a multi-well tray, a microcard, a microslide, a capillary, an etched channel plate, a molded channel plate, an embossed channel plate, etc. The sample chamber can be part of a combination of multiple sample chambers grouped into a row, an array, an assembly, etc. Multi-chamber arrays can include 12, 24, 36, 48, 96, 192, 384, 1536, 3072, 6144, or more sample chambers. The sample chamber can be shaped to a multi-well tray under the SBS microtiter format.

The term “biological sample” as used herein refers to any biological or chemical substance, typically in an aqueous solution with luminescent dye that can produce emission light in relation to nucleic acid present in the solution. The biological sample can include one or more nucleic acid sequence to be incorporated as a reactant in polymerase chain reaction (PCR) and other reactions such as ligase chain reaction, antibody binding reaction, oligonucleotide ligations assay, hybridization assay and isothermal amplification. The biological sample can include one or more nucleic acid sequence to be identified for DNA sequencing. The term “nucleic acid” as used herein refers to DNA, RNA, PNA, variations thereof, and other oligonucleotides or their analogs.

The term “luminescent dye” as used herein refers to fluorescent or phosphorescent dyes that can be excited by excitation light or chemiluminscent dyes that can be excited chemically. Luminescent dyes can be used to provide different colors depending on the dyes used. Several dyes will be apparent to one skilled in the art of dye chemistry. One or more colors can be collected for each dye to provide identification of the dye or dyes detected. The dye can be a dye-labeled fragment of nucleotides. The dye can be a marker triggered by a fragment of nucleotides. The dye can provide identification of nucleic acid sequence in the biological sample by association, for example, bonding to or reacting with a detectable marker, for example, a respective dye and quencher pair. The respective identifiable component can be positively identified by the luminescence of the dye. The dye can be normally quenched, then can become unquenched in the presence of a particular nucleic acid sequence in the biological sample or be quenched and become unquenched. The fluorescent dyes can be selected to exhibit respective and, for example, different, excitation and emission wavelength ranges. The luminescent dye can be measured to quantitate the amount of nucleic acid sequences in the biological sample. The luminescent dye can be detected in real-time to provide information about the identifiable nucleic acid sequences throughout the reaction. Examples of fluorescent dyes with desirable excitation and emission wavelengths can include 5-FAM™, TET™, and VIC™. The term “luminescence” as used herein refers to low-temperature emission of light including fluorescence, phosphorescence, electroluminescence, and chemiluminescence.

In various embodiments, color-imaging detectors can have pixel filter arrays deposited on top of gray-scale detectors. For photographic applications, red, green, or blue transmitting materials are placed in patterns with unequal numbers of each color so as to maximize perceived photopic or scotopic sharpness (“lightness or darkness”). Sharpness of this sort is desirable since it is pleasing to human viewers. However, in the detection of luminescent dyes that can produce emission light in relation to nucleic acid it is not desirable to provide unequal numbers of each color if the numbers are set to optimize photopic or scotopic sharpness. For example, patterns with more green relative to blue or red can degrade the quantitation of luminescent dyes detected. It is desirable to provide pixel filter arrays to match the luminescent dyes in the biological sample. Such matching can be achieved by predicting the relative photon flux from different luminescent dyes in a biological sample at the detection concentration (or Ct for SDS applications) of each dye. Loss of color detail can be offset by detecting in regions of interest. In various embodiments, pixels can be binned to receive the same color configuration by aligning a photomask with the pixel bins.

In various embodiments, color-imaging detectors can be multi-color pixel imaging detectors that include a single pixel area for imaging three colors. This can be achieve, for example, by placing three layers of imaging material that can provide reconstruction of red, green, and blue light at that single pixel location because different wavelengths of light travel different depths into silicon. This diverges from the traditional color imaging of using three pixels (red, blue, and green) to reconstruct the color of an image in a location by interpolating the three pixels. Such multi-color pixel imaging detectors can include the X3 from Foveon, Inc. (Santa Clara, Calif.). In various embodiments, luminescent dyes can emit light at specific wavelength spectra. The layering of the imaging material can be selected to discriminate between the different wavelength spectra of the luminescent dyes such that the amount of emission light emitted by the individual dyes can be measured. In various embodiments, each layer can provide discrimination of dye wavelengths to measure the wavelength spectrum of light emitted from a single dye. In various embodiments, each layer can provide discrimination of emitted light for dye calibration and spectral deconvoluition such that the amount of light emitted from each dye can be calculated.

In various embodiments, multiple luminescent dyes can be deconvolved spectrally to facilitate further downstream analysis. This process transforms the data from a linear combination of light emission from the multiple luminescent dyes to one in which the layers correspond to relative dye species emission intensity. In embodiments with spectral overlap, each signal in a set of spectral bands can be measured and correlated to the relative concentration of each dye of the multiple dyes using an inverted calibration matrix, in lieu of measuring the signal form a single dye of the multiple dyes. The raw data can be related to the underlying dye emission wavelengths through the matrix of dye spectral profiles. The underlying source signals can be inverted to provide the spectral deconvolution of the raw data. The optical detection system can be first calibrated to obtain the spectral calibration matrix. That matrix can be inverted in one of the signal-processing step by multicomponent transformation that is also known as spectral calibration or spectral deconvolution. Spectral calibration can include running a known set of analytes through the system in such a way that spectral regions represented by a limited number of dye species can be identified. From these regions, the spectra that characterize individual dyes can be computed to construct the matrix. A method of performing this transformation is contemplated in U.S. Pat. No. 6,333,501.

In various embodiments, instead of having only three imaging layers, the multi-color pixel imaging detectors use a single pixel area for imaging more than three colors. Such imaging layers can provide discrimination of narrower wavelength spectra thereby providing discrimination of more dye wavelength spectra.

Reference will now be made to various exemplary embodiments, examples of which are illustrated in the accompanying drawings. Wherever possible, the same reference numbers are used in the drawings and the description to refer to the same or like parts.

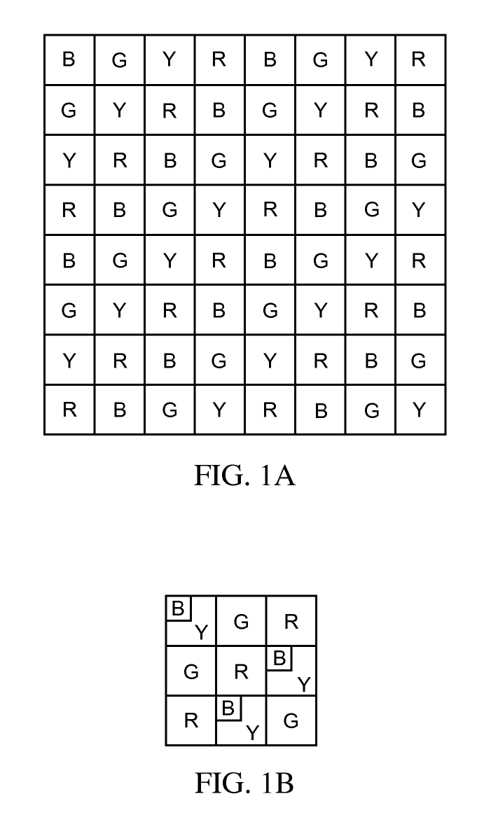

In various embodiments, a pixel filter array can include four color filters, for example, red, blue, green, and yellow to match dyes such as FAM, VIC, TAMRA, and ROX. As illustrated in FIG. 1A, the filter array 100 can be matched to the dye emissions with colors ratioed to account for relative emission strengths. As illustrated in FIG. 1B, the resulting signals can be proportional to the sum of the product of emission light, transmission, and ratio of pixels/color. The filter array illustrated in FIGS. 1A-1B can be used with a stationary sample. For example, an array of samples in a nucleic acid sequence detection instrument or an array of beads in a nucleic acid sequencing instrument.

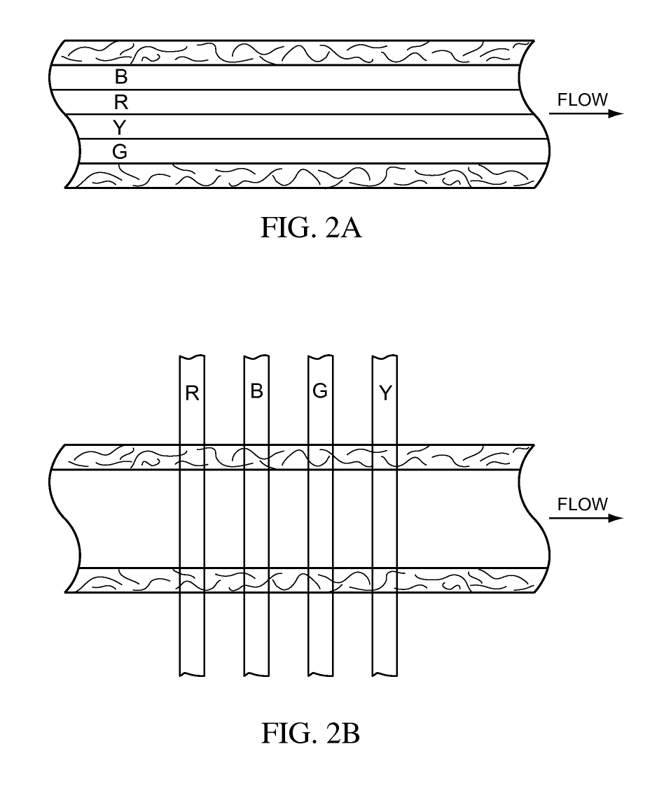

In various embodiments, a pixel filter array can be used with a moving sample, for example, a sample migrating in a capillary, as in capillary electrophoresis used for DNA sequencing. As illustrated in FIG. 2A, the filter array 100 can be oriented longitudinal such that the stripes can be parallel to the axis of the capillary image, with thickness (width) tailored for signal uniformity. As illustrated in FIG. 2B, the filter array 100 can be oriented transverse such that the stripes can be perpendicular to the axis of the capillary image, with thickness (width) tailored for signal uniformity). In various other embodiments, the filter array can be oriented such that the stripes can have any angle of incidence from zero to ninety degrees as in FIGS. 2A and 2B, for instance the angle can be 20, 45, 60, etc. degrees.

In various embodiments, the pixel filter array can be positioned at the detector. For example, the pixel filter array can be integrated into the detector. Alternatively, the pixel filter array can be positioned at an intermediate image plane between the biological samples and the detector. In this embodiment, the emission light can be imaged on the pixel filter array and the resulting filtered light can be imaged onto a detector. The imaging onto the pixel filter array and the imaging onto the detector can be provided by separate lenses or other optical configurations as known in the art of optical imaging.

In various embodiments, a method using a multi-color pixel-imaging detector can include exciting the luminescent dye with an excitation source that excites a biological sample to provide either fluorescence or phosphorescence. The method can further include providing an optical filter to prevent the excitation light from reaching the detector. The multi-color pixel-imaging detector can be custom fabricated to provide three layers whose thickness can be optimized to balance the signal-to-noise ratio and condition number for three dyes such as FAM, VIC, and ROX. In various embodiments, lack of a filter can be combined with filters, for example, red, blue emission, all excitation light, red excitation light, blue, all red light, etc. In such an embodiment, not all the pixels have filters. This is a better quantification for one color.

For the purposes of this specification and appended claims, unless otherwise indicated, all numbers expressing quantities, percentages or proportions, and other numerical values used in the specification and claims, are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements. Moreover, all ranges disclosed herein are to be understood to encompass any and all subranges subsumed therein. For example, a range of “less than 10” includes any and all subranges between (and including) the minimum value of zero and the maximum value of 10, that is, any and all subranges having a minimum value of equal to or greater than zero and a maximum value of equal to or less than 10, e.g., 1 to 5.

It is noted that, as used in this specification and the appended claims, the singular forms “a,” “an,” and “the” include plural referents unless expressly and unequivocally limited to one referent. Thus, for example, reference to “a light source” includes two or more different light sources. As used herein, the term “include” and its grammatical variants are intended to be non-limiting, such that recitation of items in a list is not to the exclusion of other like items that can be substituted or added to the listed items.

Various embodiments of the teachings are described herein. The teachings are not limited to the specific embodiments described, but encompass equivalent features and methods as known to one of ordinary skill in the art. Other embodiments will be apparent to those skilled in the art from consideration of the present specification and practice of the teachings disclosed herein. It is intended that the present specification and examples be considered as exemplary only.

What is claimed is:

1. A method for detection for a biological sample, the method comprising:
exciting multiple luminescent dyes that produce emission light in relation to nucleic acids present in the biological sample; and
detecting the emission light with a multi-color detector, wherein the detector provides dedicated sections of the detector corresponding to the multiple dyes.

2. The method of claim 1, wherein the method does not include filtering the emission light with a transmission filter for selecting luminescence wavelengths and detecting the luminescence wavelengths on a monochromatic detector.

3. The method of claim 1, wherein the multi-color detector comprises a pixel filter array.

4. The method of claim 3, wherein the pixel filter array is positioned at the detector.

5. The method of claim 3, wherein the pixel filter array is positioned at an intermediate image plane.

6. The method of claim 3, wherein the pixel filter array comprises filters corresponding to the multiple dyes, and wherein the filters match the proportion of emission light from each dye at a detection concentration for the nucleic acids.

7. The method of claim 6, further comprising thermally cycling the biological sample for amplification of the nucleic acids to reach the detection concentration.

8. The method of claim 3, wherein the pixel filter array comprises filters positioned along a flow path of the nucleic acids.

9. The method of claim 8, wherein the filters are oriented parallel to the flow path.

10. The method of claim 9, further comprising separating the nucleic acids in the biological sample by electrophoresis.

11. The method of claim 8, wherein the filters are oriented perpendicular to the flow path.

12. The method of claim 11, further comprising separating the nucleic acids in the biological sample by electrophoresis.

13. The method of claim 1, wherein detecting comprises multi-color imaging within a single pixel.

14. The method of claim 13, wherein the multi-color imaging within a single pixel comprises providing layers corresponding to each of the multiple dyes.

15. The method of claim 13, further comprising deconvolving the emission light and detecting in multiple layers.

16. The method of claim 15, further comprising thermally cycling the biological sample for amplification of the nucleic acids to reach a detection concentration.

17. The method of claim 15, further comprising separating the nucleic acids in the biological sample by electrophoresis.

18. A method for detection for a biological sample, the method comprising:
exciting multiple luminescent dyes that produce emission light in relation to nucleic acids present in the biological sample; and
detecting the emission light with a multi-color detector, wherein the method does not include filtering the emission light with a transmission filter for selecting luminescence wavelengths, and detecting the luminescence wavelengths on a monochromatic detector.

19. The method of claim 18, wherein detecting comprises discriminating the emission light from the multiple dyes.

20. The method of claim 19, wherein discriminating comprises providing a pixel filter array positioned at the detector.

21. The method of claim 19, wherein discriminating comprises providing a pixel filter array positioned at an intermediate image plane.

22. The method of claim 19, wherein discriminating comprises providing multi-color imaging within a single pixel.

23. The method of claim 19, wherein the discriminating comprises providing discrimination between chemiluminescence and fluorescence.

24. A system for detection for a biological sample, the method comprising:
means for exciting multiple luminescent dyes that produce emission light in relation to nucleic acids present in the biological sample;
means for detecting the multi-color emission light without a luminescent-band selecting filter and a monochromatic detector; wherein the means for detecting comprises a means for discriminating the multi-spectral emission light.
